(19) United States
(12) Patent Application Publication
(10) Pub. No.: US 2023/0355355 A1
(43) Pub. Date: Nov. 9, 2023

(54) REGAINER AND METHOD OF MANUFACTURING SAME

(71) Applicant: Smilecad Co., Ltd., Busan (KR)

(72) Inventors: Seok Joon KIM, Busan (KR); Jae Ho JEON, Busan (KR); Byeong Soo KIM, Busan (KR)

(73) Assignee: Smilecad Co., Ltd., Busan (KR)

(21) Appl. No.: 18/189,224

(22) Filed: Mar. 24, 2023

(30) Foreign Application Priority Data
May 6, 2022 (KR) 10-2022-0056188

(51) Int. Cl. A61C 7/14; A61C 7/00

(52) U.S. Cl. CPC A61C 7/14; A61C 7/002

(57) ABSTRACT

Proposed is a regainer including a body part which is integrally formed of a shape memory material and supports teeth at opposite sides of tooth loss space, wherein the body part includes a first grip part which covers a portion of a perimeter of a tooth disposed at a first side relative to the tooth loss space, a second grip part which covers a portion of a perimeter of a tooth disposed at a second side relative to the tooth loss space, and a bridge which connects the first grip part with the second grip part and elastically supports the first grip part and the second grip part, wherein the bridge is formed in a form bent multiple times in the tooth loss space.

### CROSS REFERENCE TO RELATED APPLICATION

The present application claims priority to Korean Patent Application No. 10-2022-0056188, filed May 6, 2022, the entire contents of which is incorporated herein for all purposes by this reference.

### BACKGROUND OF THE INVENTION

### Field of the Invention

The present disclosure relates generally to a regainer and a method of manufacturing the same. More particularly, the present disclosure relates to a regainer which is formed of a shape memory material and a method of manufacturing the same.

### Description of the Related Art

A regainer is a device for expanding and restoring space loss occurring at a location at which a tooth is lost, and is also called a space securing device.

When space loss occurs at a location at which a tooth is lost, a permanent tooth may fail to erupt or implant treatment may be difficult. Accordingly, for the eruption of the permanent tooth and the implant treatment, the lost space is required to be expanded and restored, and to this end, a regainer is applied in dental treatment.

A conventional regainer is configured to have a closed loop structure to be fitted over teeth disposed at first and second sides of tooth loss space, respectively, or is configured to have a pair of fixing parts attached to the localized parts of teeth by adhesive, a spring which applies elastic force to the pair of fixing parts so that the pair of fixing parts moves away from each other, and a coupling element for coupling the spring to the fixing parts. Each component is manufactured separately and coupled to each other by welding or the coupling element.

U.S. Patent Application Publication No. 2010/0136497 A1, which is a prior art, discloses a regainer comprising: a fixing part having two attaching surfaces attached respectively to teeth the opposite sides of tooth loss space; coupling elements of a piston structure which extend respectively from the two attaching surfaces and are coupled slidably to each other; and a coil spring which supplies elastic force to the coupling elements so that the coupling elements move away from each other.

However, to manufacture such a conventional regainer as the regainer of the prior art, after a plaster model of a patient's tooth arrangement is manufactured, a fixing part and a spring suitable for the patient's teeth are prepared and coupled to each other, and the regainer is mounted to the plaster model, a detailed adjustment process is required.

Accordingly, it takes a long time to manufacture the regainer, manufacturing costs thereof increases, and during a manufacturing period thereof, the reduction of the tooth loss space may proceed. In addition, since the configuration of the regainer is complex, it is not easy to manufacture the regainer, and there is a high possibility of quality defects such as abnormal operation of the regainer or damage to coupling portions thereof.

### DOCUMENT OF RELATED ART

(Patent Document 1) U.S. Patent Application Publication No. 2010/0136497 A1

### SUMMARY OF THE INVENTION

Accordingly, the present disclosure has been made keeping in mind the above problems occurring in the related art and is intended to propose a regainer which can be easily manufactured as an integral structure based on the scanning data of tooth arrangement and a method of manufacturing the same.

The present disclosure is intended to propose a regainer which does not require an additional treatment process to secure the mounting space of the regainer and a method of manufacturing the same.

In order to achieve the above objectives, according to one aspect of the present disclosure, there is provided a regainer including: a body part which is integrally formed of a shape memory material and supports teeth at opposite sides of tooth loss space, wherein the body part includes: a first grip part which covers a portion of a perimeter of a tooth disposed at a first side relative to the tooth loss space; a second grip part which covers a portion of a perimeter of a tooth disposed at a second side relative to the tooth loss space; and a bridge which connects the first grip part with the second grip part and elastically supports the first grip part and the second grip part, wherein the bridge is famed in a form bent multiple times in the tooth loss space.

In addition, the first grip part and the second grip part respectively may have curved shapes open in directions opposite to each other.

In addition, the bridge may be disposed along a centerline connecting centers of the teeth of the opposite sides of the tooth loss space and may be formed by being bent multiple times in a zigzag pattern in lingual and buccal side directions intersecting with the centerline.

In addition, the bridge may be located in a portion between a lingual side tangent line in contact with the first grip part and the second grip part at a lingual side and a buccal side tangent line in contact with the first grip part and the second grip part at a buccal side.

In order to achieve the above objective, according to an embodiment of the present disclosure, there is provided a method of manufacturing the regainer having the body part comprising the plurality of grip parts configured to respectively cover portions of perimeters of teeth disposed at opposite sides of reduced tooth loss space for expanding the reduced tooth loss space, and the bridge connecting the plurality of grip parts to each other and elastically supporting the plurality of grip parts, the method including: acquiring 3D scanning data of tooth arrangement including the reduced tooth loss space by using a 3D scanning device; processing the scanning data, based on which after expanding the reduced tooth loss space, a plurality of grip-part processing lines respectively covering portions of perimeters of teeth disposed at opposite sides of expanded tooth loss space, and a bridge processing line connecting the plurality of grip-part processing lines to each other are created; and laser cutting a plate-shaped base material foamed of a shape memory alloy material based on processing line data including the data on the grip-part processing lines and the bridge processing line so as to form a body-part forming body having a basic shape of the body part in the base material.

The regainer according to the embodiment of the present disclosure may be expected to have the following effects.

The body part of the regainer is manufactured by laser cutting a shape memory alloy plate or by 3D printing based on the scanning data of tooth arrangement so that the body part is formed as one integral component, thereby facilitating the manufacturing of the body part and greatly decreasing manufacturing time thereof.

In addition, a bridge connects the pair of grip parts spaced apart from each other and is formed in a shape bent multiple times in side directions so that the bridge can be provided to be stably transformable in the tooth loss space, thereby effectively preventing a user from feeling unnatural and supporting the pair of grip parts stably and elastically.

Furthermore, each of the plurality of grip parts supporting respective teeth at opposite sides relative to the tooth loss space is formed to cover a portion of the perimeter of a corresponding tooth, and particularly, is formed by being open toward a side opposite to the tooth loss space. Accordingly, unlike a conventional regainer covering the entire perimeter of a tooth, the regainer of the present disclosure makes an additional treatment process for securing the mounting space of the regainer between adjacent teeth to be unnecessary, thereby effectively solving problems such as a patient's discomfort and the increase of treatment period due to the additional treatment process.

The manufacturing method of the regainer according to the embodiment of the present disclosure may be expected to have the following effects.

The body part can be manufactured as one integral component, thereby greatly reducing the manufacturing cost of the regainer.

In addition, the body part is manufactured by laser cutting based on the 3D scanning data of tooth arrangement, thereby greatly reducing a period required for manufacturing of the regainer.

Furthermore, as time required for the manufacturing of the regainer is reduced, the reduction of the tooth loss space is minimized during the manufacturing period of the regainer, and thus the manufactured regainer and teeth are stably in agreement with each other, thereby minimizing attachment time of the regainer and teeth, and decreasing a treatment period.

Additionally, due to the characteristics of laser processing, it is difficult to form a vertical curve or inclination on a cut surface. However, in the manufacturing method of the regainer according to the embodiment of the present disclosure, after generating a processing line defining a laser cutting shape based on the 3D scanning data of tooth arrangement, laser cutting is performed according to the processing line to form the body-part forming body. After that, through a separate additional processing, a contact-surface forming surface of the body-part foaming body, which forms the tooth contact surface of the grip part, is processed in consideration of the shape of a tooth surface. Accordingly, the curve and inclination of the tooth surface are considered in foaming the tooth contact surface of the grip part so that the grip part can be more closely attached to the tooth, thereby preventing foreign matter from being stuck in teeth and greatly improving the stability of the attachment.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a view illustrating a state in which a regainer according to the embodiment of the present disclosure is attached to teeth;

FIG. 2 is a perspective view of the regainer according to the embodiment of the present disclosure;

FIG. 3 is a view illustrating a process in which tooth loss space is restored by the regainer according to the embodiment of the present disclosure;

FIG. 4 is a view illustrating the width limitation range of a bridge according to the embodiment of the present disclosure;

FIG. 5 is a flowchart illustrating the manufacturing method of the regainer according to the embodiment of the present disclosure;

FIG. 6 is a flowchart illustrating additional processing steps in detail according to the embodiment of the present disclosure; and

FIG. 7 is a view roughly illustrating the manufacturing process of the regainer according to the embodiment of the present disclosure.

### DETAILED DESCRIPTION OF THE INVENTION

Hereinafter, the embodiment of a regainer of the present disclosure will be described in detail with reference to the accompanying drawings.

However, it cannot be said that the spirit of the present disclosure is limited to the presented embodiment, and other degenerative inventions or other embodiments included in the scope of the spirit of the present disclosure may be easily presented by adding, changing, or deleting other elements.

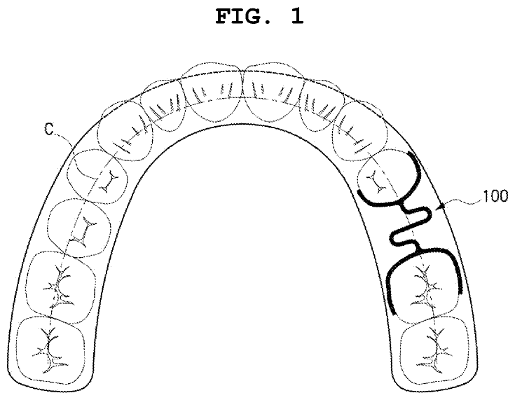

FIG. 1 is a view illustrating a state in which the regainer according to the embodiment of the present disclosure is attached to teeth.

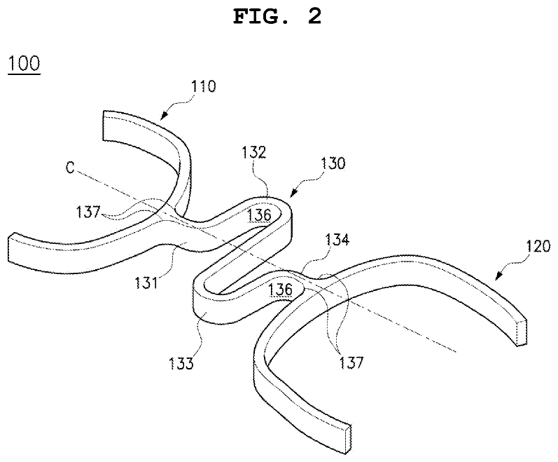

FIG. 2 is a perspective view of the regainer according to the embodiment of the present disclosure.

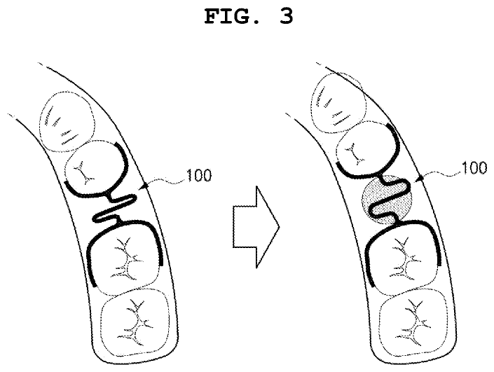

FIG. 3 is a view illustrating a process in which tooth loss space is restored by the regainer according to the embodiment of the present disclosure.

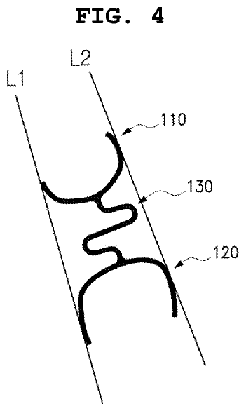

FIG. 4 is a view illustrating the width limitation range of a bridge according to the embodiment of the present disclosure.

When the reduction of the tooth loss space in which a tooth is lost occurs, the regainer 1 according to the embodiment of the present disclosure may be applied to expand and maintain the tooth loss space.

For example, when due to the shedding of a deciduous tooth prior to the eruption of permanent teeth, tooth loss space is formed and the reduction of the tooth loss space occurs, the regainer 1 may be applied to secure space for the eruption of the permanent teeth.

For another example, when teeth are permanently lost and the implantation procedure of artificial teeth is required, tooth loss space is required to be secured for the procedure, and the regainer 1 may be applied to expand reduced tooth loss space and maintain the tooth loss space.

The regainer 1 according to the embodiment of the present disclosure may include a body part 100 supporting teeth at opposite sides of tooth loss space in the arrangement of a patient's teeth.

The body part 100 may be formed as one integral component and may be famed of a shape memory material.

For example, the body part 100 may be formed of a shape memory metal material, and may be specifically formed of nitinol.

Alternatively, the body part 100 may be formed of a shape memory polymer material or a composite material having shape memory properties.

The body part 100 has a property of restoring an original shape thereof according to the characteristics of a material, and may be foamed to be elastically transformable.

The body part 100 may include: a first grip part 110 which covers a portion of the perimeter of a tooth disposed at a first side relative to tooth loss space; a second grip part 120 which covers a portion of the perimeter of a tooth disposed at a second side relative to the tooth loss space; and the bridge 130 which connects the first grip part 110 with the second grip part 120.

The first grip part 110 and the second grip part 120 may be formed to cover respective corresponding teeth in the tooth loss space.

In addition, each of the first grip part 110 and the second grip part 120 may be attached and fixed to a corresponding tooth by adhesive while covering the corresponding tooth.

Specifically, each of the first grip part 110 and the second grip part 120 may be famed to cover an inner side tooth surface facing tooth loss space, a lingual side tooth surface, and a buccal side tooth surface of a corresponding tooth.

In this case, for stability of the attachment, each of the first grip part 110 and the second grip part 120 may be formed to cover at least a half of the length of the perimeter of each of the lingual and buccal side tooth surfaces of a corresponding tooth. Each of the first grip part 110 and the second grip part 120 may be preferably famed to cover a half to two thirds of the length of the perimeter of each of the lingual and buccal side tooth surfaces of a corresponding tooth.

That is, each of the first grip part 110 and the second grip part 120 may cover a corresponding tooth in the tooth loss space, and may be foiled in a curved shape open toward a side opposite to the tooth loss space.

A first end of the bridge 130 may be connected to the first grip part 110, and a second end thereof may be connected to the second grip part 120.

In this case, the bridge 130 may be configured to be connected to a center portion of each of the first grip part 110 and the second grip part 120.

The center portion of each of the first grip part 110 and the second grip part 120 may be regarded as a part corresponding to the center of the tooth loss space in a width direction thereof intersecting with the direction of the tooth arrangement.

Specifically, a line extending along the center of the tooth arrangement of FIG. 1 may be defined as a centerline C.

The centerline C may be regarded as a line connecting the centers of arranged teeth to each other.

Based on the centerline C, the center portion of each of the first grip part 110 and the second grip part 120 may be defined as a portion at which the center portion meets the centerline C.

The bridge 130 may extend to pass through an approximate center of the tooth loss space, and may be formed in a structure that is bent multiple times in directions intersecting with the longitudinal direction of the tooth loss space.

That is, the bridge 130 extends from the center portion of the first grip part 110 toward the center portion of the second grip part 120, and may extend by bending multiple times in directions intersecting with a direction of the center portion of the first grip part 110 toward the center portion of the second grip part 120.

More specifically, the bridge 130 may include: a first extension part 131 extending from the center portion of the first grip part 110 toward the center portion of the second grip part 120; a second extension part 134 extending from the center portion of the second grip part 120 toward the center portion of the first grip part 110; and an elastic part which connects the first extension part 131 with the second extension part 134 and bends multiple times.

The first extension part 131 may protrude from the first grip part 110 along the centerline C.

The second extension part 134 may protrude from the second grip part 120 along the centerline C.

The first extension part 131 and the second extension part 134 may be located on the same straight line.

The elastic part may be configured to provide elasticity in order to secure the tooth loss space.

When the first grip part 110 and the second grip part 120 come closer to each other from preset respective initial positions, the elastic part may be elastically transformed. In addition, the elastic part may provide elastic force so that the first grip part 110 and the second grip part 120 move away from each other in the elastically transformed state.

The elastic part may be foamed in a structure bent multiple times to be curved in an approximate “S” shape.

More specifically, the elastic part is disposed along the centerline C and may include a first elastic part 132 and a second elastic part 133 which protrude in directions opposite to each other relative to the centerline C.

After the first elastic part 132 extends from the end part of the first extension part 131 in a first side direction intersecting with the first extension part 131, the first elastic part 132 may bend to be curved in a second side direction opposite to the first side direction, and may extend to the centerline C.

In addition, after the second elastic part 133 extends from the end part of the first elastic part 132 in the second side direction, the second elastic part 133 may bend to be curved in the first side direction opposite to the second side direction, may extend to the centerline C, and may be connected to the end part of the second extension part 134.

That is, the first elastic part 132 and the second elastic part 133 may be respectively formed in approximate “U” shapes by protruding in the side directions opposite to each other relative to the centerline C.

Since each of the first elastic part 132 and the second elastic part 133 is formed in an approximate “U” shape, each of the first elastic part 132 and the second elastic part 133 may have transformation space 136 for elastic transformation.

A portion at which the elastic part is connected with the first extension part 131 and a portion at which the elastic part is connected with the second extension part 134 may be formed to be curved.

That is, the first elastic part 132 may bend to be curved in the first side direction intersecting with the first extension part 131 from the end part of the first extension part 131.

In addition, the second elastic part 133 may also bend to be curved in the second side direction intersecting with the second extension part 134 from the second extension part 134.

Meanwhile, in the embodiment of the present disclosure, the elastic part is illustrated to include one elastic part, but is not limited to the embodiment of the present disclosure. The elastic part may include a plurality of elastic parts formed between the first extension part 131 and the second extension part 134.

Meanwhile, the elastic part may have a limited width so as not to protrude outside the tooth loss space.

That is, the elastic part may be limited in a width so that the elastic part is prevented from protruding to a lingual side and a buccal side out of the tooth loss space and causing a user unnatural feeling or discomfort.

For example, referring to FIG. 4, a straight line in contact with the outer sides of the first grip part 110 and the second grip part 120 at the lingual side may be defined as a lingual side tangent line L1, and a straight line in contact with the outer sides of the first grip part 110 and the second grip part 120 at the buccal side may be defined as a buccal side tangent line L2.

In addition, the elastic part may have a limited width so that the elastic part is located in a region between the lingual side tangent line L and the buccal side tangent line L2.

Meanwhile, the body part 100 as a whole may be formed to have thickness and height of 0.3 mm or more and 0.55 mm or less. That is, the section of the grip part 110 or 120 and the bridge 130 may have width and height within the range of 0.3 mm to 0.55 mm.

Meanwhile, the end part of the bridge 130 in contact with the grip part 110 or 120 may be formed to have increased thickness so that a sharp concave edge is not famed between the bridge 130 and the grip part.

Specifically, the end part of the bridge 130 in contact with the grip part 110 or 120 may have a curved rib 137 formed by being curved to opposite sides. In this case, the thickness of the end part of the bridge 130 may be increased so that the thickness of the end part of the bridge 130 does not exceed 0.55 mm.

Accordingly, a connection part at which the grip part 110 or 120 is in contact with the bridge 130 does not have a sharp concave edge, so the connection part can be prevented from being broken. In addition, the bridge 130 may support the grip part more stably, and the pressing force of the bridge 130 may be more effectively transmitted to the grip part.

Meanwhile, the body part 100 of the regainer according to the embodiment of the present disclosure may be manufactured of a shape memory material based on 3D scanning data of tooth arrangement, and various methods capable of manufacturing the above-described regainer of the present disclosure as one integral component may be applied.

For example, the regainer may be manufactured of a shape memory material by 3D printing based on the 3D scanning data of tooth arrangement.

Specifically, by using design tools such as CAD and CAM, a 3D model of the regainer may be designed by considering the tooth loss space at a position at which the regainer is required to be installed and the shapes and arrangements of teeth neighboring to the tooth loss space in the 3D scanning data of tooth arrangement.

In addition, the regainer corresponding to the 3D model of the regainer may be printed and be manufactured with a shape memory material by using a 3D printer.

The shape memory material used in the 3D printer may use various shape memory materials available for a known 3D printer, and a technique of printing an object corresponding to a 3D model by using a 3D printer and a shape memory material is disclosed in various known technologies, so detailed descriptions thereof will be omitted.

Alternatively, the regainer may be manufactured by being laser cut from a base material of a shape memory alloy material based on the 3D scanning data of tooth arrangement.

In addition, the body part 100 may be manufactured by applying additional processing for shape transformation after laser cutting a shape memory alloy material.

In addition, the body part 100 may be manufactured after a shape memory alloy material is laser cut and heat-treated. Accordingly, the body part 100 may have a higher hardness and may have a stabilized shape.

In addition, the body part 100 may be manufactured by polishing a surface thereof. Accordingly, the body part 100 may be formed to have a smoother surface, and sharp parts thereof may be removed to form a safe overall structure.

Hereinafter, a method of manufacturing the regainer according to the embodiment of the present disclosure by the laser cutting of the base material of a shape memory alloy material will be described in detail with reference to the drawings.

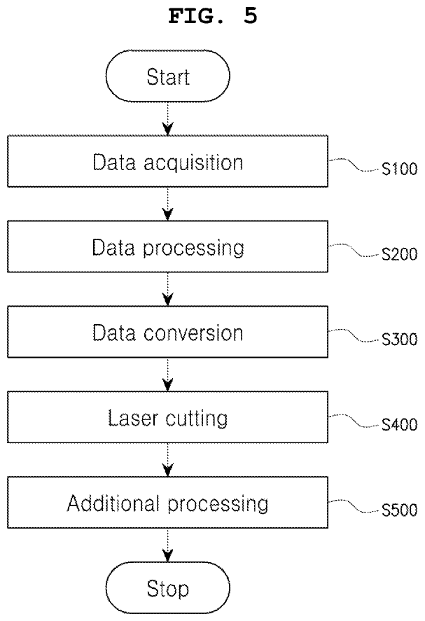

FIG. 5 is a flowchart illustrating the manufacturing method of the regainer according to the embodiment of the present disclosure.

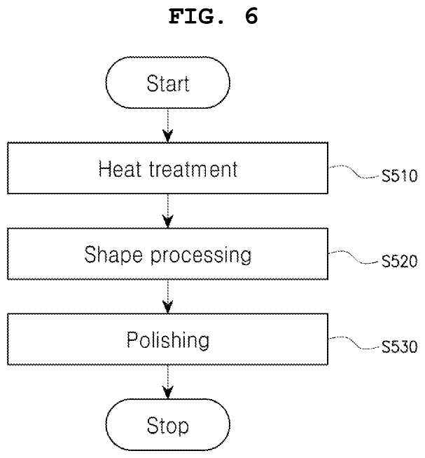

FIG. 6 is a flowchart illustrating additional processing steps in detail according to the embodiment of the present disclosure.

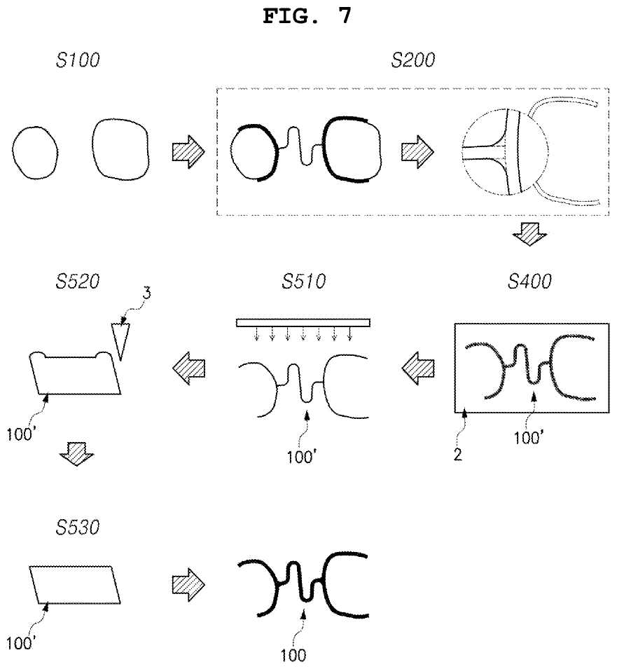

FIG. 7 is a view roughly illustrating the manufacturing process of the regainer according to the embodiment of the present disclosure.

The method of manufacturing the regainer according to the embodiment of the present disclosure may include: acquiring the scanning data of tooth arrangement including the tooth loss space; processing the scanning data; converting modified data to data for laser processing; laser cutting the base material; and performing additional processing of a body-part foaming body formed by the laser cutting.

First, the 3D scanning data of tooth arrangement including the position of an affected portion to which the regainer is required to be applied may be obtained by using a 3D scanning device.

In this case, the scanning data refers to captured data obtained by capturing the arrangement of teeth of an upper or lower jaw, to which the body part 100 will be attached, by using a 3D camera.

The scanning data may include information on the tooth loss space and tooth arrangement.

In addition, the scanning data may further include information on the curve and inclination of a tooth surface for each tooth.

The scanning data may be a file corresponding to an stl file format. [S100, data acquisition] After the data acquisition, in order to manufacture the body part 100, data processing for creating a processing line defining a laser cutting shape based on the scanning data may be performed.

After acquiring the scanning data, the tooth loss space may be expanded so that the space intended for treatment can be secured by correcting a reduced tooth loss space based on the scanning data.

In addition, it is possible to create a pair of grip-part processing lines for defining the cutting shapes of the first grip part 110 and the second grip part 120 on teeth disposed at opposite sides relative to the expanded tooth loss space based on the corrected scanning data.

Each of the grip-part processing lines may be drawn along the inner side tooth surface facing the tooth loss space, the lingual side tooth surface, and the buccal side tooth surface of a corresponding tooth. The grip-part processing lines may be created by including a pair of processing lines spaced apart from each other side by side to correspond to thickness required for presetting the thickness.

In addition, the centerline C, the lingual side tangent line L1, and the buccal side tangent line L2 may be created based on the corrected scanning data.

In addition, a bridge processing line for defining the cutting shape of the bridge 130 may be created by connecting the pair of grip-part processing lines to each other.

The bridge processing line may be disposed along the centerline C and may be formed in a form bent multiple times in directions intersecting with the centerline C.

In addition, the bridge processing line may be created to be located within an expanded portion between the lingual side tangent line L1 and the buccal side tangent line L2.

The bridge processing line may be created by including a pair of processing lines spaced apart from each other side by side to correspond to thickness required for presetting the thickness of the bridge 130.

In addition, the end part of the bridge processing line in contact with the grip-part processing line may be processed to be curved on opposite sides of the end part so that the rib 137 can be formed.

In the data processing, since the grip-part processing line is drawn along the tooth surface of a corresponding tooth, the grip-part processing line may be created in consideration of the shape of the tooth surface of the tooth. [S200, the data processing] When the data processing is completed, data of the created processing line may be converted into the data for laser processing.

For example, the processing line data may be stored as two-dimensional data.

In addition, the processing line data may be converted into the data for laser processing by using a program that automatically creates a moving path of a laser cutter based on the two-dimensional data.

The created data for laser processing may be a two-dimensional CAD file in which the processing line data is considered. [S300, conversion of data]

When the convention of data is completed, the data for laser processing may be input to the laser cutter for laser processing.

In addition, according to the data for laser processing, the laser cutter may operate to laser cut the base material 2 which is input. That is, based on the data for laser processing, the laser cutter may be regarded to laser cut the base material 2.

Specifically, the base material 2 may be provided to have the shape of a flat plate made of a shape memory alloy material. That is, the base material 2 may be a shape memory alloy plate. For example, the base material 2 may be a thin nitinol plate.

The laser cutter may be provided with a jig on which the base material 2 is seated and is fixed. In addition, the base material 2 may be seated and fixed on the jig.

The laser cutter may receive preset values before laser cutting. For example, an operator may preset values such as processing speed of 200 mm/s, processing acceleration of 3000 mm/s, pulse-type pulse power of 300 w, pulse frequency of 600 hz, pulse time of 400 us, and argon gas pressure of 3.0 mpa, etc.

After the base material 2 is input and the preset values of the laser cutter are preset, an operator may operate the laser cutter.

By cutting the base material 2 along the processing line by the laser cutter when the laser cutter operates, the body-part forming body 100′ having the basic shape of the body part 100 may be famed. [S400, laser cutting]

After the laser cutting is performed, the additional processing of the base material 2 of which the body-part forming body 100′ is formed may be performed.

In the additional processing, mechanical processing or chemical processing may be performed so that the body-part forming body 100′ has the final shape of the body part 100.

For example, the additional processing may include a heat treatment for stabilizing the shape of the body-part forming body 100′.

In addition, the additional processing may include a shape processing which applies the shape of a tooth surface to a contact-surface forming surface of the body-part forming body 100′ which constitutes the contact surface of the grip part in contact with the tooth surface.

In addition, the additional processing may include polishing the body-part foaming body 100′ in which the shape processing is completed.

The heat treatment, the shape processing, and the polishing are not limited to an order in which operations thereof are listed, and may be changed in various ways according to operation efficiency. For example, after the shape processing, the polishing may be performed, and after the polishing, the heat treatment may be performed. Alternatively, after the heat treatment is performed after the shape processing, the polishing may be performed.

Hereinafter, one example in which after the heat treatment, the shape processing is performed and after the shape processing, the polishing is performed will be described in detail. [S500, the additional processing] After the body-part forming body 100′ is cut from the base material 2, the body-part forming body 100′ may be fixed in the cut space of the base material 2 or in a separate jig, and then put into a heat treatment device to be heat treated.

In order to stabilize the shape of the body-part forming body 100′ after the body-part forming body 100′ is heat treated, the body-part forming body 100′ may be cooled by being put into a cooling device or may be cooled naturally. [S510, the heat treatment]

The body-part foaming body 100′ may be put into the shape processing after being cut from the base material 2.

The shape processing may be pertained by various tools or devices capable of cutting the surface of the body-part forming body 100′. For example, the shape processing may be performed by a cone-shaped bur 3.

Due to the characteristics of laser processing, it may be difficult to form a vertical curve or inclination on a cut surface. Accordingly, the contact-surface forming surface of the body-part taming body 100′ may be processed to have a curve or inclination by using a cutting tool or device in consideration of the shape of a tooth surface.

For example, the contact-surface forming surface of the body-part forming body 100′ may be processed by using the bur 3.

In this case, the contact-surface forming surface of the body-part forming body 100′ may be processed according to the shape of a tooth surface based on the scanning data.

For example, when the contact-surface forming surface of the body-part forming body 100′ corresponds to a tooth surface inclined upward toward the tooth loss space, the contact-surface taming surface may be processed in a form inclined downward toward the corresponding tooth surface.

In addition, when the contact-surface forming surface of the body-part forming body 100′ corresponds to a tooth surface inclined downward toward the tooth loss space, the contact-surface forming surface may be processed in a form inclined upward toward the corresponding tooth surface.

In addition, when the contact-surface forming surface of the body-part forming body 100′ corresponds to a curved tooth surface, the contact-surface foaming surface may be processed to be curved to correspond to the curved tooth surface. [520, the shape processing]

After the shape processing is performed, the polishing of the body-part forming body 100′ may be performed.

In the polishing, the body-part forming body 100′ may be polished by various polishing tools or devices capable of polishing the surface of the body-part forming body 100′.

A method of polishing a plate of a shape memory alloy is variously disclosed in prior arts, so detailed description thereof will be omitted.

The body part 100 may be formed to have a smoother surface by the polishing, and due to the removal of sharp parts, may be formed to have a safe structure as a whole.

Specifically, during the laser cutting, flash may be formed on the body-part foaming body due to melting of the base material. In addition, during the shape processing, an iron thorn may be formed in a processed portion.

Flash, iron thorns, and corners are processed by the polishing so that the surface of the body-part forming body 100′ can be formed to be more smooth and sharp parts thereof can be removed. [S530, the polishing]

According to the regainer according to the embodiment of the present disclosure described above, the body part of the regainer is manufactured by laser cutting a shape memory alloy plate or by 3D printing based on the scanning data of tooth arrangement so that the body part is formed as one integral component, thereby facilitating the manufacturing of the body part and greatly decreasing manufacturing time thereof.

In addition, the bridge connects the pair of grip parts spaced apart from each other and is formed in a shape bent multiple times in side directions so that the bridge can be provided to be stably transformable in the tooth loss space, thereby effectively preventing a user from feeling unnatural and supporting the pair of grip parts stably and elastically.

In addition, each of the plurality of grip parts supporting respective teeth at opposite sides relative to the tooth loss space is formed to cover a portion of the perimeter of a corresponding tooth, and particularly, is formed by being open toward a side opposite to the tooth loss space. Accordingly, unlike a conventional regainer covering the entire perimeter of a tooth, the regainer of the present disclosure makes an additional treatment process for securing the mounting space of the regainer between adjacent teeth to be unnecessary, thereby effectively solving problems such as a patient's discomfort and the increase of treatment period due to the additional treatment process.

Meanwhile, according to the manufacturing method of the regainer according to the embodiment of the present disclosure described above, the body part 100 can be manufactured as one integral component, thereby greatly reducing the manufacturing cost of the regainer.

In addition, the body part 100 is manufactured by laser cutting based on the 3D scanning data of tooth arrangement, thereby greatly reducing a period required for manufacturing of the regainer.

In addition, as time required for the manufacturing of the regainer is reduced, the reduction of the tooth loss space is minimized during the manufacturing period of the regainer, and thus the manufactured regainer and teeth are stably in agreement with each other, thereby minimizing attachment time of the regainer and teeth, and decreasing a treatment period.

In addition, due to the characteristics of laser processing, it is difficult to form a vertical curve or inclination on a cut surface. However, in the manufacturing method of the regainer according to the embodiment of the present disclosure, after generating a processing line defining a laser cutting shape based on the 3D scanning data of tooth arrangement, laser cutting is performed according to the processing line to form the body-part forming body 100′. After that, through a separate additional processing, the contact-surface forming surface of the body-part forming body 100′, which forms the tooth contact surface of the grip part, is processed in consideration of the shape of a tooth surface. Accordingly, the curve and inclination of the tooth surface are considered in forming the tooth contact surface of the grip part so that the grip part can be more closely attached to the tooth, thereby preventing foreign matter from being stuck in teeth and greatly improving the stability of the attachment.

What is claimed is:

1. A regainer comprising:
a body part which is integrally formed of a shape memory material and supports teeth at opposite sides of tooth loss space,
wherein the body part comprises:
a first grip part which covers a portion of a perimeter of a tooth disposed at a first side relative to the tooth loss space;
a second grip part which covers a portion of a perimeter of a tooth disposed at a second side relative to the tooth loss space; and
a bridge which connects the first grip part with the second grip part and elastically supports the first grip part and the second grip part, wherein the bridge is famed in a form bent multiple times in the tooth loss space.

2. The regainer of claim 1, wherein the first grip part and the second grip part respectively have curved shapes open in directions opposite to each other.

3. The regainer of claim 1, wherein the bridge is disposed along a centerline connecting centers of the teeth of the opposite sides of the tooth loss space and is formed by being bent multiple times in a zigzag pattern in lingual and buccal side directions intersecting with the centerline.

4. The regainer of claim 1, wherein the bridge is located in a portion between a lingual side tangent line in contact with the first grip part and the second grip part at a lingual side and a buccal side tangent line in contact with the first grip part and the second grip part at a buccal side.

5. A method of manufacturing a regainer having a body part comprising a plurality of grip parts configured to respectively cover portions of perimeters of teeth disposed at opposite sides of reduced tooth loss space for expanding the reduced tooth loss space, and a bridge connecting the plurality of grip parts to each other and elastically supporting the plurality of grip parts, the method comprising:
acquiring 3D scanning data of tooth arrangement comprising the reduced tooth loss space by using a 3D scanning device;
processing the scanning data, based on which after expanding the reduced tooth loss space, a plurality of grip-part processing lines respectively covering portions of perimeters of teeth disposed at opposite sides of expanded tooth loss space, and a bridge processing line connecting the plurality of grip-part processing lines to each other are created; and
laser cutting a plate-shaped base material formed of a shape memory alloy material based on processing line data comprising the data on the grip-part processing lines and the bridge processing line so as to form a body-part forming body having a basic shape of the body part in the base material.
